(19) United States
(12) Patent Application Publication
(10) Pub. No.: US 2013/0144743 A1
(43) Pub. Date: Jun. 6, 2013

(54) OPHTHALMIC LENS STORING UNIT WITH PROGRAMMABLE AND COMMUNICATION ELEMENTS FOR MONITORING THE USE AND AUTOMATED ORDERING

(71) Applicants: Randall B. Pugh, Jacksonville, FL (US); Edward R. Kernick, Jacksonville, FL (US); Terry O'Brien, Jacksonville, FL (US); Karson S. Putt, Jacksonville, FL (US); James Daniel Riall, St. Johns, FL (US); Andres F. Arrubla, Jacksonville, FL (US)

(72) Inventors: Randall B. Pugh, Jacksonville, FL (US); Edward R. Kernick, Jacksonville, FL (US); Terry O'Brien, Jacksonville, FL (US); Karson S. Putt, Jacksonville, FL (US); James Daniel Riall, St. Johns, FL (US); Andres F. Arrubla, Jacksonville, FL (US)

(21) Appl. No.: 13/370,669

(22) Filed: Feb. 10, 2012

Related U.S. Application Data
(63) Continuation-in-part of application No. 13/312,167, filed on Dec. 6, 2011.

(51) Int. Cl. G06Q 30/00

(52) U.S. Cl. 705/26.1

(57) ABSTRACT

The present invention provides for a programmable processor in a Ophthalmic Lens Storage Unit capable of storing and disinfecting ophthalmic lenses. A base unit and/or storage case can include sensors for providing data and a digital storage for storing the data and using it via executable software for lens monitoring and automated lens ordering. In some embodiments, the processor is additionally operative via the executable software to provide disinfecting radiation base radiation for disinfecting an ophthalmic lens.

### RELATED APPLICATIONS

This application is a continuation-in-part of U.S. patent application Ser. No. 13/312,167 which was filed on Dec. 6, 2011 and entitled “OPHTHALMIC LENS DISINFECTING BASE UNIT WITH PROGRAMMABLE AND COMMUNICATION ELEMENTS,” the contents of which are relied upon and incorporated by reference.

### FIELD OF USE

This invention describes a case for storing, disinfecting, and monitoring the use of an ophthalmic lens and, more specifically, in some embodiments, programmable and communication elements of a base capable of generating an automated order and sending an order through a network, for said ophthalmic lenses.

### BACKGROUND

It is well known that contact lenses can be used to improve vision. Various contact lenses have been commercially produced for many years. Early designs of contact lenses were fashioned from hard materials. Although these lenses are still currently used in some applications, they are not suitable for all patients due to their poor comfort and relatively low permeability to oxygen. Later developments in the field gave rise to soft contact lenses, based upon hydrogels.

Hydrogel contact lenses are very popular today. These lenses are often more comfortable to wear than contact lenses made of hard materials. Many hydrogel contact lenses may be worn for more than one day. However, a build-up of microbial life and bacteria on the lenses generally makes it desirable to periodically remove the lenses and disinfect them.

Disinfection of contact lenses traditionally entails placing the contact lens in a container or case and subjecting the contact lens to a chemical disinfectant. However, chemical disinfectants are not always as efficacious as may be desired. From time to time, a contact lens with a bacterium, mold, fungus or other type of adverse life form is reinserted into a user's eye with the result being a diseased eye.

New methods and approaches are therefore needed to monitor the use and order contact lenses based on a plurality of factors, such as for example, remaining use cycles, present lens conditions, eye care practitioner appointment data, and inventory of said lenses.

### SUMMARY

Accordingly, the present invention includes an Ophthalmic Lens Storage Unit with various programming and communicating options to monitor the use of ophthalmic lenses and generate an optimized automated order. In some embodiments, the lens storage case is capable of storing reusable contact lenses, disinfecting the lenses during the storage using disinfecting radiation to kill unwanted bacteria, viruses, molds, fungi and the like on a contact lens, and monitoring the condition and the use of said lenses.

In some embodiments, the Ophthalmic Lens Storage Unit can include logic to record and analyze data, to control functions of the ophthalmic lens disinfecting unit, monitor lens use and to display or cause a network associated device to send relevant messages to one or both the user and a third party.

The present invention can include one or more sensor for measuring and storing data descriptive of a condition of the lens. For example, the sensors can measure the optical properties and track the optical performance of the lens to generate a message that alerts the user that the lens must be replaced.

Additionally, in some embodiments a bar code reader can be included to allow the scanning of the package in which the lenses are contained. Upon the scanning of the barcode in the packaging, the disinfecting unit can use a network to download data corresponding to those lenses. For example, data can include, the number of lenses included in package, the recommended length of time or wearing cycles the lenses can be used before replacement, origin and lot number for the lenses, prescription information, etc.

In other aspects of the present invention, the processor and sensors can additionally be used to monitor the use and conditions of a lens before and after use. For example, a light emitter and detector can be used to measure the optical effect of the lens. The optical effect can be measured prior, during and after disinfecting of the lens is completed to monitor the number of particles that built up in the lens during wear and the effect they have in the lens performance and during cleaning. Changes in particle buildup and conditions of the lens can also be stored to detect unusual changes and then alert the user to prevent things, such as for example, an eye infection.

Finally, in some embodiments of the present invention the disinfecting unit can include a touch screen that serves as a user interface to allow the programming of preferences associated with the functionality of the device and the mode of operation of the device.

15

### DESCRIPTION OF THE DRAWINGS

FIG. 1 illustrates method steps for collecting and storing user operation data according to some embodiments of the present invention.

FIG. 2 illustrates method steps for collecting and storing sensor data according to some embodiments of the present invention.

FIG. 3 illustrates method steps for running a process or action, thereby controlling case functions, according to some embodiments of the present invention.

FIG. 4 illustrates method steps for displaying user messages according to some embodiments of the present invention.

FIG. 5 illustrates method steps for programming an Ophthalmic Lens Storage Unit.

FIG. 6 illustrates method steps for communicating data between an Ophthalmic Lens Storage Unit and other devices and entities.

FIG. 7 illustrates a processor that may be used in some embodiments of the present invention.

FIG. 8 illustrates a network diagram with devices that may be used for Automated Ordering in some embodiments of the present invention.

FIG. 9 illustrates exemplary screen shots of the touch screen display, of a disinfecting unit device depicting possible user options of the device.

FIG. 10 illustrates exemplary screen shots of a disinfecting base unit depicting a pre-cleaned lens and cleaned lens as measured by sensors that may be incorporated in an Ophthalmic Lens Storage Unit.

FIG. 11 illustrates exemplary screen shots of a disinfecting unit device depicting some of the user settings to customize the functionality of an Ophthalmic Lens Storage Unit.

FIG. 12 illustrates exemplary screen shots of a disinfecting base unit device associated with Eye Care Practitioner Appointments and Automated Lens Ordering.

### DETAILED DESCRIPTION OF THE INVENTION

The present invention includes methods and apparatus for storing and analyzing data related to disinfecting an ophthalmic lens, for controlling functions of an ophthalmic lens storage unit, and for displaying relevant user messages. Also included are options for programming an ophthalmic lens storage unit. Further, communication options allow data to be communicated between an ophthalmic lens storage unit and external devices or entities for various purposes. Such as, for example, for facilitating new orders and tracking the lenses.

In the following sections detailed descriptions of embodiments of the invention will be given. The descriptions of both preferred and alternative embodiments are exemplary embodiments only, and it is understood by those skilled in the art that variations, modifications, and alterations may be apparent. It is therefore to be understood that said exemplary embodiments do not limit the scope of the underlying invention.

### Glossary

In this description and claims directed to the presented invention, various terms may be used for which the following definitions will apply:

Associated Third Party: as used herein refers to a 3^{rd} party which may access or send information to the storage unit. For example, it can include an eye care practitioner's office, a contact lens manufacturer, or a retailer.

Authenticating Feature: as used herein refers to readable feature in a lens used to prevent counterfeiting products from being used by a user. For example, the readable feature may be a structural feature, a printed symbol or code.

Automated Order: as used herein refers to an order generated by a processor based on a plurality of factors, including for example, lens use frequency, condition of used lens, user preferences, third party input data, and number of lenses purchased.

Disinfecting Radiation: as used herein refers to a frequency and intensity of radiation sufficient to diminish the life expectancy of a life form receiving a Disinfecting Radiation Dose.

Disinfecting Radiation Dose: as used herein refers to an amount of radiation to reduce an amount of life by at least two logs on a logarithmic scale and preferably three logs or more, wherein life includes at least bacteria, viruses, molds and fungi.

Lens: refers to any ophthalmic device that resides in or on the eye. These devices can provide optical correction or may be cosmetic. For example, the term lens can refer to a contact lens, intraocular lens, overlay lens, ocular insert, optical insert or other similar device through which vision is corrected or modified, or through which eye physiology is cosmetically enhanced (e.g. iris color) without impeding vision. In some embodiments, the preferred lenses of the invention are soft contact lenses made from silicone elastomers or hydrogels, which include but are not limited to silicone hydrogels, and fluorohydrogels.

Ophthalmic Lens Storing Unit: as used herein refers to a disinfecting base unit, disinfecting storage case, or a combination thereof, capable of storing reusable contact lenses and disinfecting the lenses during the storage. For example, by through the use of a solution and/or receiving disinfecting radiation in a wavelength and intensity suitable to kill unwanted bacteria, viruses, molds, fungi and the like on a contact lens. In some embodiments, the ophthalmic lens storage unit can include more than one storage cases for disinfecting.

Radiation Disinfecting Base Unit: as used herein refers to a device capable of receiving one or more Radiation Disinfecting Storage Cases to provide disinfecting radiation in wavelengths, durations, and intensities suitable to kill unwanted bacteria, viruses, molds, fungi and the like on one or more contact lenses, and in some embodiments, additionally on the surfaces of the disinfecting storage.

Radiation Disinfecting Storage Case: as used herein refers to a lens storage case capable of storing reusable contact lenses and disinfecting the lenses during the storage by receiving disinfecting radiation in a wavelength and intensity suitable to kill unwanted bacteria, viruses, molds, fungi and the like on a contact lens.

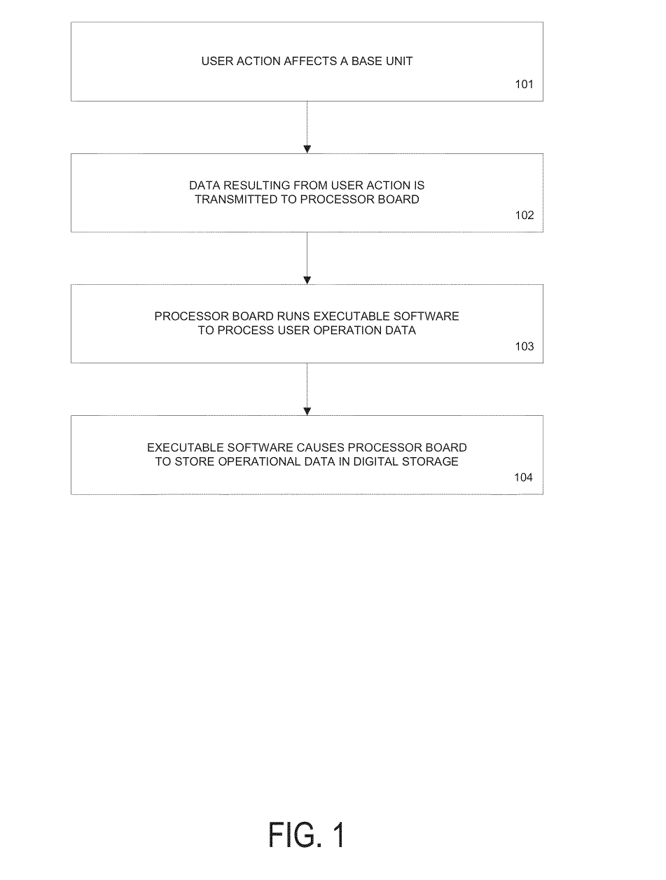

Referring now to FIG. 1, a flowchart illustrates exemplary steps that may be used to implement the present invention. At 101, a user performs an action that affects a radiation disinfecting base unit of an ophthalmic lens disinfecting unit. An action may include, for example, opening a radiation disinfecting base unit, closing a radiation disinfecting base unit, inserting a radiation disinfecting storage case, removing a radiation disinfecting storage case, or pressing a button.

At 102, data resulting from a user action is transmitted to a processor board. Data transmission may include direct electrical connection, such as, for example, via a universal serial buss (USB) or via a wireless transmission, such as for example a radio frequency transmission (RF transmission), Bluetooth, or other mechanism for logical communication.

At 103, a processor board runs executable software to process data resulting from a user's action. In some embodiments, executable software resets a lens disinfecting cycle counter after a user presses a reset button on a radiation disinfecting base unit to indicate that fresh lenses are being used. In additional embodiments, executable software resets a radiation disinfecting storage case timer after a user presses a reset button on a radiation disinfecting base unit to indicate a radiation disinfecting storage case has been replaced. Other embodiments include, by way of non-limiting example, executable software incrementing counters for cleaning cycles, timers for lens and storage case usage, and other functions associated with lens and storage case use and disinfection.

At 104, executable software causes a processor board to store data in digital storage. Stored data may include data based on user actions, measurements from sensors, as well as changes resulting from executable software functions such as resetting counters and timers. In some preferred embodiments, stored data includes a date and time associated with a user action or with an executable software action. Data storage may include, for example, user preferred settings, storage in one or more of a historical data log, a current lens data log, and a current radiation disinfecting storage case data log.

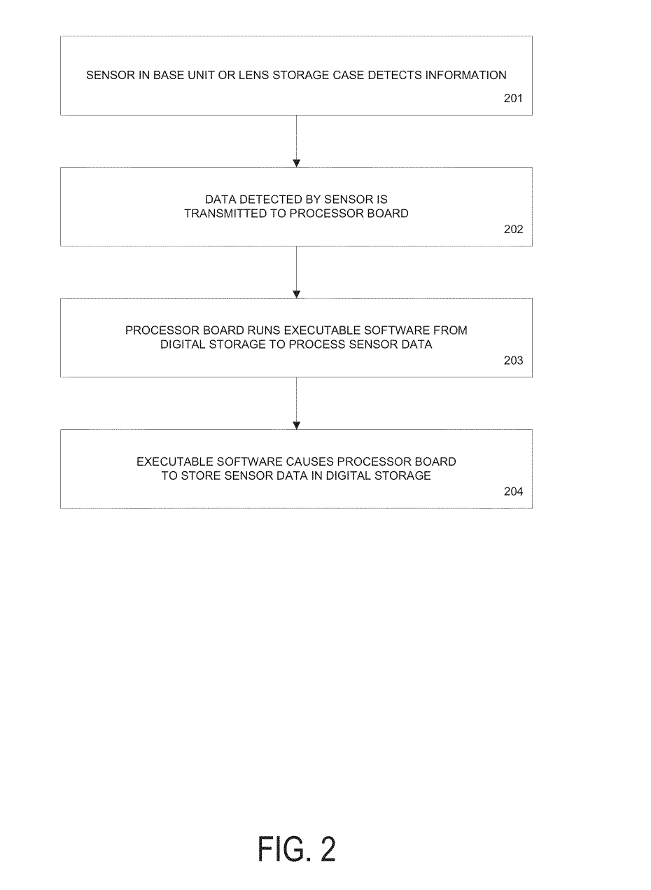

Referring now to FIG. 2, a flowchart illustrates exemplary steps that may be used to implement additional aspects of the present invention. At 201, a sensor in a radiation disinfecting base unit or a radiation disinfecting storage case detects information. A sensor may include, for example, an LED sensor, a charged couple device (CCD) array, a CCD camera, a barcode scanner, or other known sensor such as, for example a device to detect one or both of a an intensity light and a time duration of exposure to a particular wavelength of radiation, temperature of solution in which the lens is stored, pH of solution in which the lens is stored, moisture, amount of protein build up in the lens, or other condition. At 202, sensor data is transmitted to a processor board. Data transmission may include direct electrical connection, radio frequency transmission, or other mechanism for logical communication or transmission. Although the term transmitted is used to describe the transfer of data from the sensor to the processor board, data may be polled from the sensor or otherwise communicated. Each respective transmission medium will be accompanied by an appropriate transmission device. For example, an RF transmission will include a RF transmitter located within the storage case and a RF receiver in the base. Preferred embodiments include both an RF transmitter and receiver in the base and storage case. A direct electrical communication will include a conductive path between the sensor in the storage case and the processor in the base.

At 203, a processor board runs executable software to process sensor data. In some embodiments, sensor data is compared to historical data to determine contact lens or storage case cleanliness. Various embodiments may also include comparison of sensor data to stored baseline data to detect if a radiation disinfecting storage case is present within a radiation disinfecting base unit and to detect if contact lenses are present within a radiation disinfecting storage case. In additional embodiments, sensor data is compared to stored lens profile data, uniquely identifying a contact lens brand. In still other embodiments, sensor data is compared to stored lens data to detect the optical power of each contact lens and thereby identify the right contact lens and the lens contact lens to assist the user of the disinfecting unit.

For example, in some embodiments, a predetermined amount of ultraviolet (UV) radiation may be passed through a contact lens stored in the storage case. One or more sensors may be used to detect amounts of UV radiation passing through one or more portions of the contact lens. A profile may be generated of amounts of radiation passing through the one or more portions. Particular types of lenses will generate identifiable patterns in the profiles. The patterns may be used to identify a type of lens, or even a specific lens.

At 204, executable software causes a processor board to store data in digital storage. Stored data may include data collected by sensors as well as data resulting from executable software analysis such as, for example, a number of days until lenses should be replaced, a number of days until a storage case should be replaced, a percentage of lens opacity indicating lens cleanliness, a percentage of radiation disinfecting storage case opacity indicating radiation disinfecting storage case cleanliness, presence or absence of radiation disinfecting storage case, presence or absence of contact lenses, identified lens brand, and identified lens optical powers. In some preferred embodiments, stored data includes a date and time associated with sensor data or with results from executable software. Data storage may include, for example, storage in one or more of a historical data log, a current lens data log, a current radiation disinfecting storage case data log, and a sensor-specific data log.

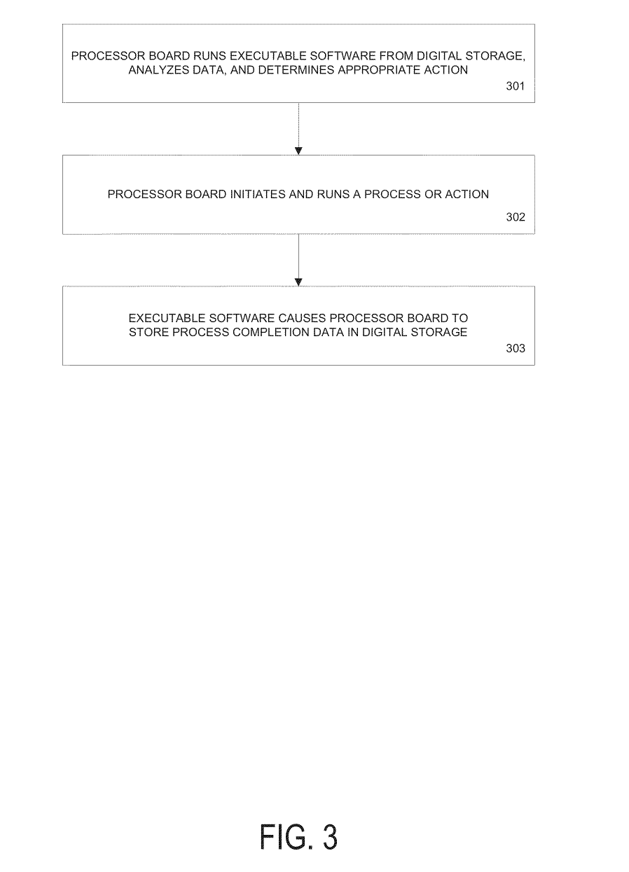

Referring now to FIG. 3, a flowchart illustrates exemplary steps that may be used to implement additional aspects of the present invention. At 301, a processor board runs executable software to analyze previously stored data and determine an appropriate action. In some embodiments of the present invention, executable software analyzes whether a radiation disinfecting base unit has recently been plugged in or been closed. In other embodiments, executable software analyzes stored data to determine when a cleaning cycle should be started. Specific details such as the duration, pattern, and intensity of disinfecting radiation used in a cleaning cycle are calculated or selected in some embodiments of the present invention. In other aspects of the present invention, executable software identifies whether a radiation disinfecting base unit is currently plugged into an external power source.

At 302, a processor board runs a process or action, such as by way of non-limiting example, a radiation disinfecting base case initialization routine and a radiation disinfecting cleaning cycle. Other embodiments include power management actions such as charging a battery in a radiation disinfecting base unit, running the base unit from battery power or running the base unit from direct power.

At 303, executable software causes a processor board to store process completion data in digital storage. Process completion data may include, for example, data related to case initialization processes, including detection of LED strength, detection of presence of radiation disinfecting storage case, detection of contact lenses within radiation disinfecting storage case, and detection of correct contact lens powers in each well of a radiation disinfecting storage case. In other aspects of the present invention, stored data is related to a radiation disinfecting cleaning cycle including duration of radiation, pattern of radiation timing, radiation intensity, and post-disinfection cleanliness data pertaining to contact lenses and radiation disinfecting storage case. In still other embodiments, completion data includes battery charging time, percent battery full, time periods in which base case was operated using a battery and in which base case was operated using direct power. In some preferred embodiments, stored data includes a date and time associated with process completion data. Data storage may include, for example, storage in one or more of a historical data log, a current lens data log, and a current radiation disinfecting storage case data log.

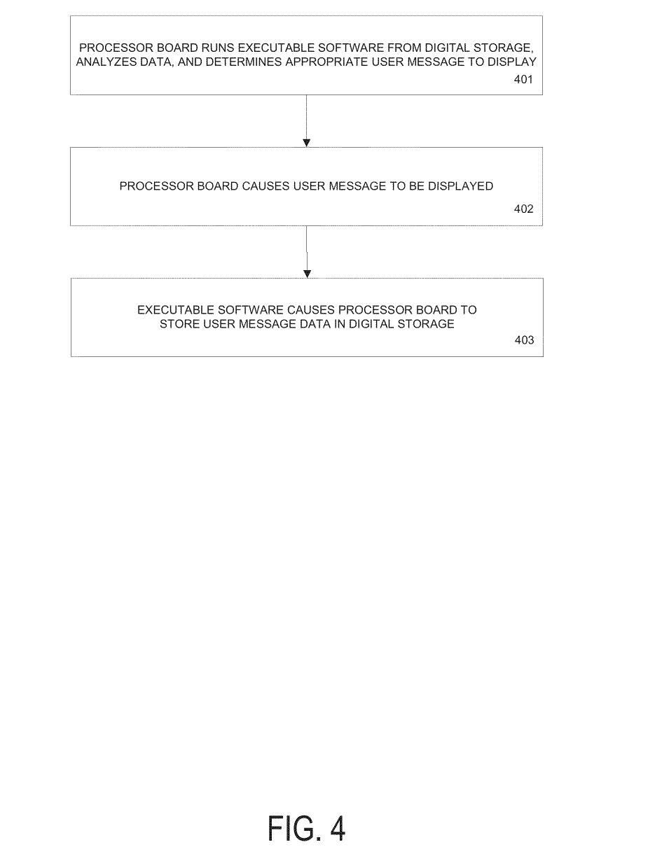

Referring now to FIG. 4, a flowchart illustrates exemplary steps that may be used to implement additional aspects of the present invention. At 401, a processor board runs executable software to analyze previously stored data and determines an appropriate user message to be displayed. In some embodiments of the present invention, executable software analyzes, for example, the remaining life of the lens, the need for an appointment with an eye care practitioner, remaining lenses, ordering information and automated ordering, tracking of lenses using a barcode to ensure lenses are not counterfeit lenses, and recommendations of new products for the specific user.

At 402, a processor board causes a user message to be displayed on a message display area of a radiation disinfecting base unit. Some embodiments include messages indicating it is time to insert and disinfect contact lenses, it is time to begin using a new pair of contact lenses, it is time to begin using a new radiation disinfecting storage case, it is time to make an annual appointment with the user's eye care professional, it is time to order new lenses, and new product information specific to the user. In other embodiments, warning messages are displayed, for example, a disinfecting cycle was interrupted, a disinfecting cycle did not complete properly, bar code is not recognized/match the lens per the database and the user should contact a customer service representative, a user has mixed up their right and left contact lenses by placing lenses in the wrong wells of a radiation disinfecting storage case, a user should see their eye care professional soon, as for example, when an unusually high buildup of microbes on lenses has been detected. Still other embodiments include base unit status messages such as, for example, current battery level, battery needs to be recharged, there is not enough battery to complete a cleaning cycle so unit must be plugged in, battery can no longer be recharged so it is time to replace the radiation disinfecting base unit, one or more LEDs are decaying so it is time to replace the radiation disinfecting base unit. Further embodiments include instructional messages such as how to resume a disinfecting cycle, how to restart a disinfecting cycle, how to reprogram a radiation disinfecting base unit, how to store data on a computer or other external device, and how to send data to an eye care professional or other party.

At 403, executable software causes a processor board to store user message data in digital storage. User message data may include, for example, an indication of a specific message displayed and a reason that triggered the specific message. For example, a message to change a contact radiation disinfecting storage case may be triggered because a time limit has been reached or because a sensor detected a change indicator on a radiation disinfecting storage case. In some preferred embodiments, stored data includes a date and time associated with user message display. Data storage may include, for example, storage in one or more of a historical data log, a current lens data log, and a current radiation disinfecting storage case data log.

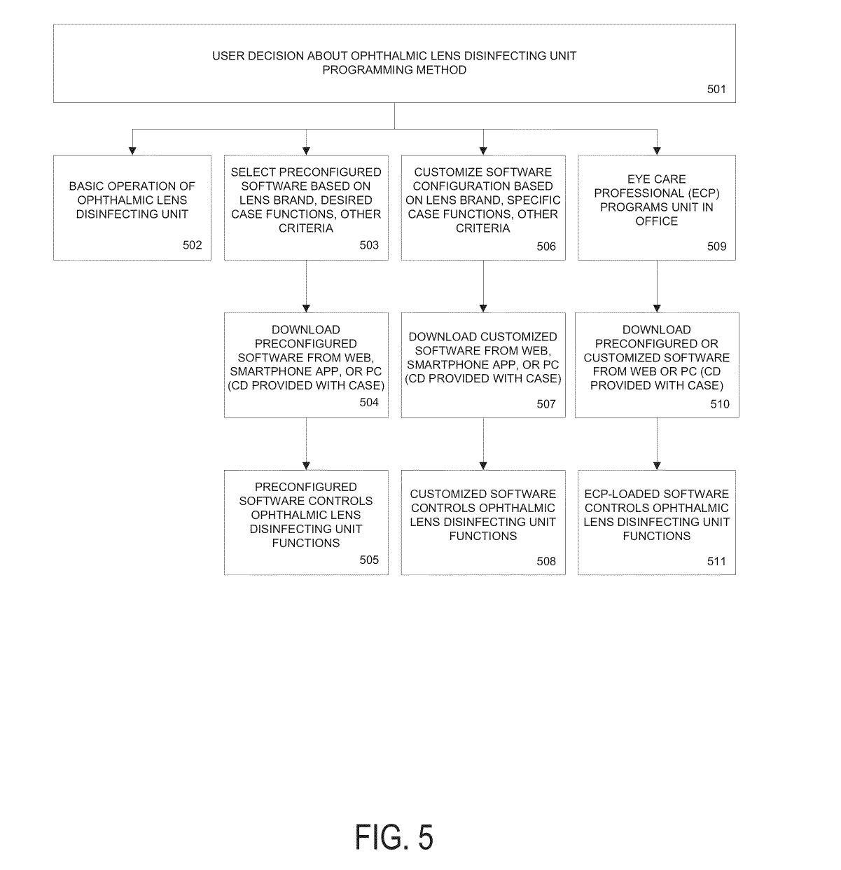

Referring now to FIG. 5, a flowchart illustrates exemplary steps that may be used to implement additional aspects of the present invention. At 501, a user determines how to program an ophthalmic lens disinfecting unit by selecting specific setting options. The user decision may be based on information provided with an ophthalmic lens disinfecting unit, information available on a website, information from their eye care professional, information from lens manufacturer or other source. The user decision leads to 502, 503, 506 or 509. A user may download different preconfigured or customized software at any time. A new software download may be desirable if a user's lens brand or lens parameters change, if updated software is available to correct program errors, if the user desires more or less functionality from the ophthalmic lens disinfecting unit, and for other reasons. After loading a preconfigured or customized program, a user may or may not be provided with an option to revert their ophthalmic lens disinfecting unit to its basic operational state.

At 502, a user decides to use only basic functions provided with an ophthalmic lens disinfecting unit. No additional steps, programming, or configuration are necessary to use the unit with base functionality. Basic operation may include, by way of non-limiting example, initialization routine when user closes case, generic radiation disinfecting cycle appropriate for many disposable lenses, and basic user messages.

At 503, a user selects from a limited number of preconfigured software options for an ophthalmic lens disinfecting unit. Preconfigured software may be selected, for example, on the basis of the lens brand worn by the user, on desired case functions, and other criteria. At 504, selected preconfigured software is downloaded to an ophthalmic lens disinfecting unit from a website, a Smartphone application, a PC using a CD provided with an ophthalmic lens disinfecting unit, or other known method of software distribution. At 505, an ophthalmic lens disinfecting unit is controlled using preconfigured software. In addition to the functions available in basic operation, preconfigured software may support, by way of non-limiting example, radiation disinfecting cycles specific to a contact lens brand and wear schedule, counters and reminders based on standard lens wear schedules, ability to later upload data from ophthalmic lens disinfecting unit for analysis, expanded user messages, and other functions.

At 506, a user customizes software configuration for an ophthalmic lens disinfecting unit. Software may be customized, for example, by selecting specific brand and lens parameters worn by a user for each eye, by selecting custom lens wear schedules, by selecting or blocking functions such as counters and reminders, by entering date of last eye exam or lens purchase to enable reminder for subsequent eye exams or lens purchases, by entering a name or other indentifying information, by selecting data communication options, by scanning a bar code in the packaging of the lens to download specific information about the lens, and other functions. At 507, customized software is downloaded to an ophthalmic lens disinfecting unit from a website, a Smartphone application, a PC using a CD provided with an ophthalmic lens disinfecting unit, or other known method of software distribution. At 508, an ophthalmic lens disinfecting unit is controlled using customized software. In addition to the functions available in basic operation, customized software may support, by way of non-limiting example, radiation disinfecting cycles specific to a contact lens brand and parameters worn by a user, counters and reminders based on lens wear schedule indicated by a user, ability to identify wrong contact lens powers in wrong wells of storage case, ability to later upload data from ophthalmic lens disinfecting unit for analysis, customized user messages, display of user name or other identifying information, communication of data to other devices, and other functions.

At 509, an eye care professional (ECP) programs an ophthalmic lens disinfecting unit in the office for a user. An ECP may use preconfigured software as described in the path starting at step 503, or may use customized software configuration as described in the path starting at step 506. At 510, preconfigured or customized software is downloaded to an ophthalmic lens disinfecting unit from a website, a Smartphone application, a PC using a CD provided with an ophthalmic lens disinfecting unit, or other known method of software distribution. At 511, an ophthalmic lens disinfecting unit is controlled using preconfigured software as described previously in step 505, or customized software as described previously in step 508.

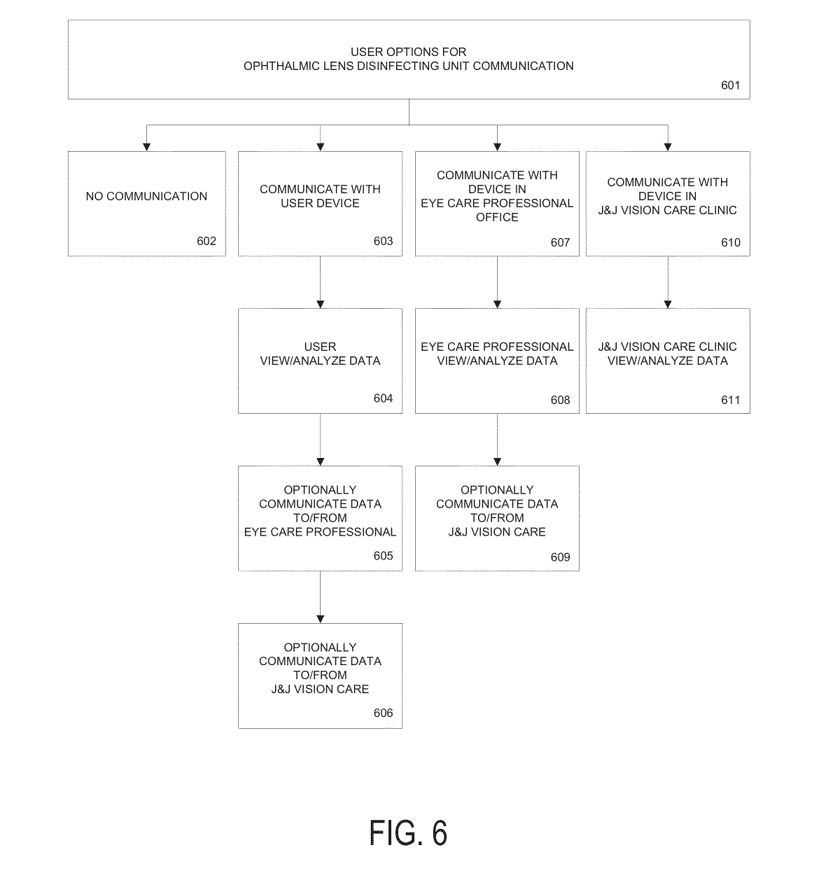

Referring now to FIG. 6, a flowchart illustrates exemplary steps that may be used to implement additional aspects of the present invention. At 601, a user may make a decision about communicating data from an ophthalmic lens disinfecting unit. Alternatively, a default communication may be implemented The user decision may be based, for example, on information provided with an ophthalmic lens disinfecting unit, information available on a website, information from their eye care professional, or other source. One or both of a user decision and a default communication mode eventually can lead to 602, 603, 607 or 610.

At 602, a user decides to use an ophthalmic lens disinfecting unit in a stand-alone manner. In various implementations, data may be stored for subsequent analysis or simply not recorded.

At 603, a user enables communication between an ophthalmic lens disinfecting unit and a user device such as a PC, Smartphone, or other device capable of receiving ophthalmic lens disinfecting unit data. In some preferred embodiments, an ophthalmic lens disinfecting unit application available via a PC, Smartphone or other device facilitates the sharing of data. An ophthalmic lens disinfecting unit application may be a standalone application running on a computing device, may be an application available on a website accessed using a computing device, or may be a distributed application with functions running on a both standalone computing device and a website.

At 604, an ophthalmic lens disinfecting unit application is used to view and analyze ophthalmic lens disinfecting unit data. In some embodiments, data includes text, charts, graphs, and other representations. Data may include, by way of non-limiting example, contact lens brand and parameters, contact lens wear schedules, user specific information, cleaning cycle details, contact lens cleanliness data, contact lens and radiation disinfecting storage case replacement history, number of days until recommended contact lens replacement, number of days until recommended radiation disinfecting storage case replacement. Other embodiments include ophthalmic lens disinfecting unit diagnostic information, such as, for example, battery information, LED information, sensor information, and communication details.

At 605, a user makes a decision to send ophthalmic lens disinfecting unit data to an eye care professional. In some preferred embodiments, an ophthalmic lens disinfecting unit application facilitates the sharing of data with an eye care professional using known data transmission methods, such as, for example, sending an email containing text information, sending an email containing a data file, making a file or data available on a website to which the eye care professional has access, or other means. Ophthalmic lens disinfecting unit data shared with an eye care professional may include, by way of non-limiting example, contact lens brand and parameters, contact lens wear schedules, user specific information, cleaning cycle details, contact lens cleanliness data, contact lens and radiation disinfecting storage case replacement history, number of days until recommended contact lens replacement, number of days until recommended radiation disinfecting storage case replacement. Other embodiments may include the sharing of ophthalmic lens disinfecting unit diagnostic information, such as, for example, battery information, LED information, sensor information, and communication details. Shared data may be in the form of text, charts, graphs, and other representations. In another aspect, in some embodiments, an eye care professional may also be capable of transmitting messages and data to a user's ophthalmic lens disinfecting unit application, where it may be viewed in the application or may be transmitted to an ophthalmic lens disinfecting unit and displayed on the display area. Eye care professional messages and data may include, for example, eye exam reminders, contact lens purchase reminders, sale information, ordering information, or other information.

At 606, a user makes a decision to send ophthalmic lens disinfecting unit data to a contact lens manufacturer or other provider of contact lenses. In some preferred embodiments, an ophthalmic lens disinfecting unit application facilitates the sharing of data with a manufacturer or other provider of contact lenses using known data transmission methods, such as, for example, sending an email containing text information, sending an email containing a data file, making a file or data available on a website to which the manufacturer or other provider of contact lenses has access, or other means. Ophthalmic lens disinfecting unit data shared with a manufacturer or other provider of contact lenses may include, by way of non-limiting example, contact lens brand and parameters, contact lens wear schedules, user specific information, cleaning cycle details, contact lens cleanliness data, contact lens and radiation disinfecting storage case replacement history, number of days until recommended contact lens replacement, number of days until recommended radiation disinfecting storage case replacement. Other embodiments may include the sharing of ophthalmic lens disinfecting unit diagnostic information, such as, for example, battery information, LED information, sensor information, and communication details. Shared data may be in the form of text, charts, graphs, and other representations. In another aspect, in some embodiments, a manufacturer or other provider of contact lenses may also be capable of transmitting messages and data to a user's ophthalmic lens disinfecting unit application, where it may be viewed in the application or may be transmitted to an ophthalmic lens disinfecting unit and displayed on the display area. Manufacturer messages and data may include, for example, eye exam reminders, contact lens purchase reminders, ordering information, and interphase for automated ordering, contact lens rebate information, contact lens purchase coupons, or other information.

At 607, a user provides an ophthalmic lens disinfecting unit to an eye care professional, who enables communication from the ophthalmic lens disinfecting unit to a device in the eye care professional office such as a PC, Smartphone, or other device capable of receiving ophthalmic lens disinfecting unit data. In some preferred embodiments, an ophthalmic lens disinfecting unit application available via a PC, Smartphone or other device facilitates the sharing of data. An ophthalmic lens disinfecting unit application may be a standalone application running on a computing device, may be an application available on a website accessed using a computing device, or may be a distributed application with functions running on a both standalone computing device and a website. At 608, an ophthalmic lens disinfecting unit application is used to view and analyze ophthalmic lens disinfecting unit data. In some embodiments, data includes text, charts, graphs, and other representations. Data may include, by way of non-limiting example, contact lens brand and parameters, contact lens wear schedules, user specific information, cleaning cycle details, contact lens cleanliness data, contact lens and radiation disinfecting storage case replacement history, number of days until recommended contact lens replacement, number of days until recommended radiation disinfecting storage case replacement. Other embodiments include ophthalmic lens disinfecting unit diagnostic information, such as, for example, battery information, LED information, sensor information, and communication details. In another aspect, in some embodiments, an eye care professional may also be capable of transmitting messages and data to a user's ophthalmic lens disinfecting unit, where it may be displayed on the display area. Eye care professional messages and data may include, for example, eye exam reminders, contact lens purchase reminders, sale information, or other information.

At 609, an eye care professional makes a decision to send ophthalmic lens disinfecting unit data to a contact lens manufacturer or other provider of contact lenses. In some preferred embodiments, an ophthalmic lens disinfecting unit application facilitates the sharing of data with a manufacturer or other provider of contact lenses using known data transmission methods, such as, for example, sending an email containing text information, sending an email containing a data file, making a file or data available on a website to which the manufacturer or other provider of contact lenses has access, or other means. Ophthalmic lens disinfecting unit data shared with a manufacturer or other provider of contact lenses may include, by way of non-limiting example, contact lens brand and parameters, contact lens wear schedules, user specific information, cleaning cycle details, contact lens cleanliness data, contact lens and radiation disinfecting storage case replacement history, number of days until recommended contact lens replacement, number of days until recommended radiation disinfecting storage case replacement. Other embodiments may include the sharing of ophthalmic lens disinfecting unit diagnostic information, such as, for example, battery information, LED information, sensor information, and communication details. Shared data may be in the form of text, charts, graphs, and other representations.

At 610, a user provides an ophthalmic lens disinfecting unit to a manufacturer or other provider of contact lenses eye care professional, who enables communication from the ophthalmic lens disinfecting unit to a device in the manufacturer office such as a PC, Smartphone, or other device capable of receiving ophthalmic lens disinfecting unit data. In some preferred embodiments, an ophthalmic lens disinfecting unit application available via a PC, Smartphone or other device facilitates the sharing of data. An ophthalmic lens disinfecting unit application may be a standalone application running on a computing device, may be an application available on a website accessed using a computing device, or may be a distributed application with functions running on a both standalone computing device and a website. At 611, an ophthalmic lens disinfecting unit application is used to view and analyze ophthalmic lens disinfecting unit data. In some embodiments, data includes text, charts, graphs and other representations. Data may include, by way of non-limiting example, contact lens brand and parameters, contact lens wear schedules, user specific information, cleaning cycle details, contact lens cleanliness data, contact lens and radiation disinfecting storage case replacement history, number of days until recommended contact lens replacement, number of days until recommended radiation disinfecting storage case replacement. Other embodiments include ophthalmic lens disinfecting unit diagnostic information, such as, for example, battery information, LED information, sensor information, and communication details. In another aspect, in some embodiments, a manufacturer or other provider of contact lenses may also be capable of transmitting messages and data to a user's ophthalmic lens disinfecting unit where it may be displayed on the display area. Manufacturer messages and data may include, for example, eye exam reminders, contact lens purchase reminders, contact lens rebate information, contact lens purchase coupons, or other information.

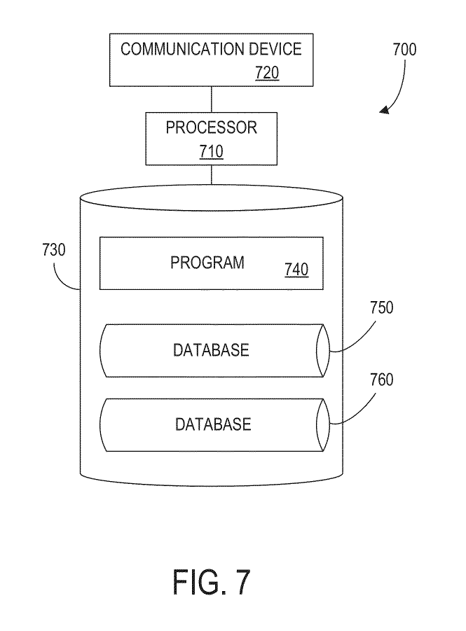

Referring now to FIG. 7 a controller 700 that may be used in some embodiments of the present invention is illustrated. The controller 700 includes a processor 710, which may include one or more processor components coupled to a communication device 720. The communication device 720 may also be configured to communicate information via a communication channel to electronically transmit and receive digital data related to the functions discussed herein.

The communication device 720 may also be used to communicate, for example, with one or more human readable display devices, such as, for example: an LCD panel, a LED display or other display device or printer. In some preferred a touch screen is utilized providing a human interface with the disinfecting base unit.

The processor 710 may also be in communication with a storage device 730. The storage device 730 may comprise any appropriate information storage device, including combinations of magnetic storage devices (e.g., magnetic tape, radio frequency tags, and hard disk drives), optical storage devices, and/or semiconductor memory devices such as Random Access Memory (RAM) devices and Read-Only Memory (ROM) devices.

The storage device 730 can store a program 740 for controlling the processor 710. The processor 710 performs instructions of the program 740, and thereby operates in accordance with the present invention. For example, the processor 710 may receive information descriptive of lens, lens inventory, eye care practitioner's office prescription or appointment information, user's preferences, and the like. The storage device 730 can also store patient related data received, from sources such as the eye care practitioner's office, directly from the manufacture's network, a third party, or imputed by the user, in one or more databases 750 and 760.

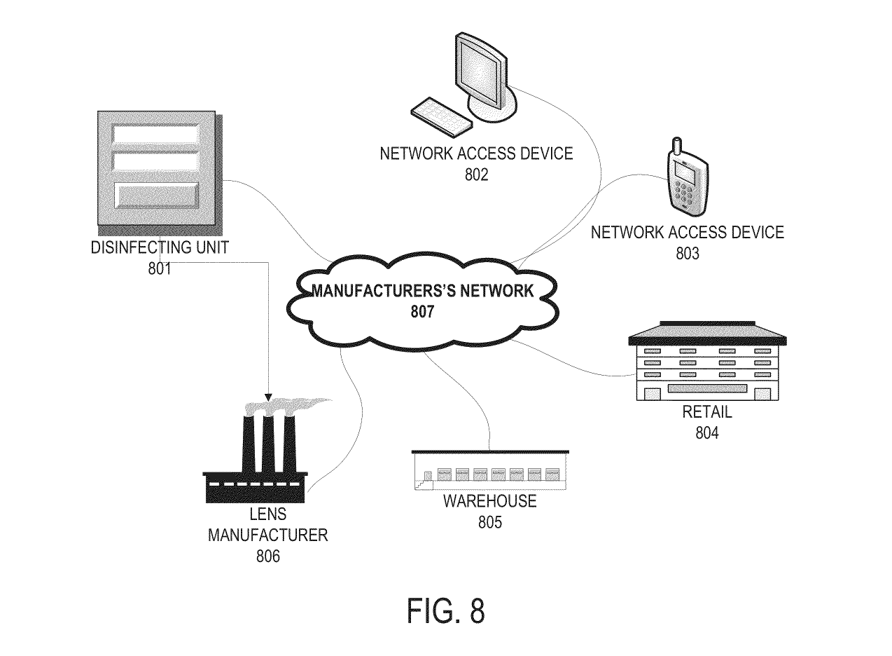

Referring now to FIG. 8, a network diagram illustrates how an Ophthalmic Lens Storage Unit 801 may be connected via a the manufacture's network 807, such as, for example, the Internet, a cellular link, a virtual private network or other vehicle for transferring digital data. The Ophthalmic Lens Storage Unit may include sensors, communicating devices, and programmed capabilities such as the ones described in this application. In addition, the apparatus may include a programmable processor and storage device connected to a human readable display device, as discussed further below in regard to FIG. 7.

Generally, the Ophthalmic Lens Storage Unit 801 can be functional to track an inventory accessible by the Ophthalmic Lens Storage Unit 801 and to generate an automated order for ophthalmic lenses based on a plurality of stored inputs in the memory. The automated order can be programmed to be sent along with payment information, for example to a manufacturer plant, or retailer upon the confirmation by the user. In some preferred embodiments, the memory in the disinfecting base unit 801 may include information about the user, user's eye care practitioner, user's purchased lenses, number of lenses remaining, weather and user's prescription. Furthermore, when lenses are received by the user, a bar code in the packaging may be scanned with some disinfecting base units to provide digital data descriptive of the lenses/order. Digital data may be processed by storing the data within the disinfecting base unit 801 or transmitted to a destination 802-806 connected to the distributed network 807. This data can be useful to track specific orders of lenses, prevent any use of counterfeit lenses which can be harmful to the user, track user feedback pertaining to the specific lenses, facilitate the recall of any specific product by alerting the user before any negative result can occur, ensure compliance with eye care practitioner's prescribed product, and track effects of weather conditions with specific lenses. In addition, weather information and allergy alerts data may be downloaded by the device for message generation and data analysis. Data analysis can include for example, correlating weather and allergy information with abnormal protein or microbial buildup.

A destination 802-806 can include, for example, a network access device 802 that includes a display and keyboard accessing the distributed network 807 under the control of a processor. The network access device may therefore include a personal computer, mobile device, laptop, or terminal. Other network access devices 803 may be mobile in nature and include personal digital assistants (“PDA”s), cellular phones with network access capabilities, net books, or other relatively small processor run appliances with the ability to send and receive digital data across a network.

Still other embodiments may include network access devices 804-805 associated with a retail location for ophthalmic lenses and solutions, a warehouse for ophthalmic lenses and solutions and an ophthalmic lens and solutions manufacturing facility.

The disinfecting base unit 801 may therefore receive information about ophthalmic lenses and solutions to assist the user of the device in tracking the use of the lenses, placing automated orders directly to the manufacturing facility, tracking eye care practitioner (“ECP”) appointments, and comparing bar code data and lens compliance to detect and signal counterfeit lenses that may not meet health standards and cause significant eye problems. For example, digital data descriptive of which manufacturing plant of the lenses, expiration dates, and optical power specifications. The automated ordering may also be transmitted from the disinfecting base unit 801 and to any network access device 802-806. The digital data may be transmitted one or both of: with no artificial delay introduced (real time), or on a periodic basis.

In some embodiments, the Ophthalmic Lens Storage Unit 801 compiles a report descriptive of an aggregate of lenses used by the user and transmits the report to network access devices associated with related entity. The related entity can be, for example, a lens manufacturer, a market analyst, an ophthalmic lens retailer; an ophthalmic lens warehouse, or other interested entity.

In some embodiments, a disinfecting base unit 801 may be functional to analyze data specific to the user and suggest things such as, for example, more trial lenses and solutions to a patient and also initiate an order for commercially available lenses. Initiation of an order may include transmission of digital data descriptive of the patient and lenses and solutions to be shipped to the patient. The digital information initiating may therefore include, for example, any or all of the following: patient name and address, billing information, payment information, lens SKU, quantity of lenses, eye care practitioner providing prescription for the lenses, weather and allergy data and any other information generally received by an online retail site for lens sales. One exemplary online sales vehicle includes Acuvue-Direct™ which processes orders for ophthalmic lenses via a distributed network 807 which includes the Internet.

In another aspect, a Ophthalmic Lens Storage Unit 801 may be functional to initiate an automated reminder, such as ACUMINDER™, to replace the patient's contact lenses after the time of recommended wear has been completed. A schedule may commence based upon the type of lens dispensed and a suggested lens wearing schedule or based on data gathered from measurement of the sensors in the disinfecting base unit. The disinfecting base unit 801 may then automatically display a reminder in the user interphase or cause to transmit a reminder, such as, for example, via one or more of: email, text message, social media, RSS, and telephone. The reminder can include a human interpretable message that instructs them to do a specific action, such as replace their lenses.

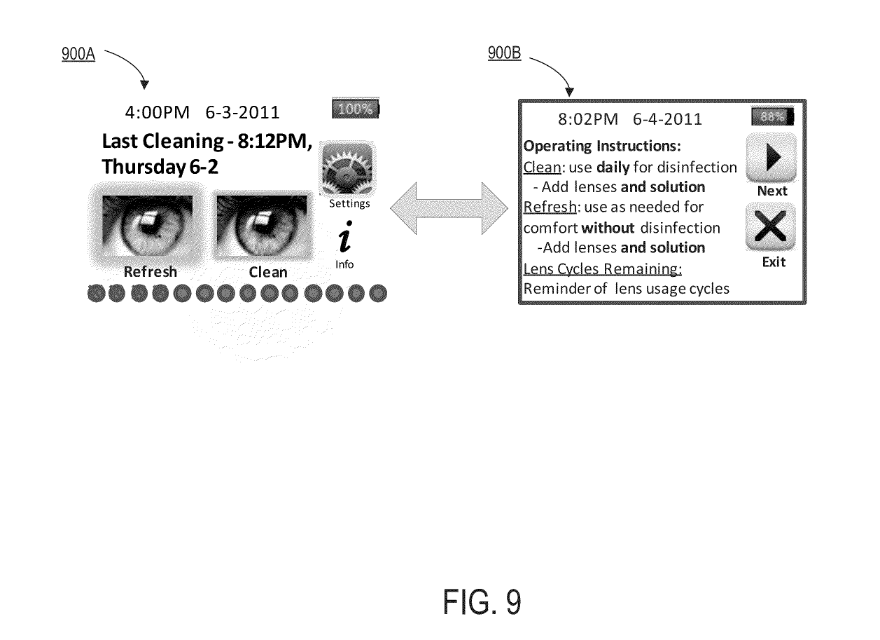

Referring now to FIG. 9, exemplary screen shots of the touch screen display of a

Ophthalmic Lens Storage Unit are depicted. Screen shot 900A of the disinfecting base unit display depicts exemplary options a user would see in the display for the lens disinfecting cycle. The software in the disinfecting base unit can show the user when his last cleaning was and how many cycles remain in the lifetime for the specific lens. Additionally, the user can see the battery life remaining in the disinfecting base unit, user settings, which in some embodiments control the disinfection process to a specified time by changing the intensity of the UV and time of exposure, and other functions as they may be programmed in the device. Further, more information about each process, instructional information, or informative data may be provided to the user. For example, as depicted in screen shot 900B operating instructions that can include different functionality of the disinfecting unit.

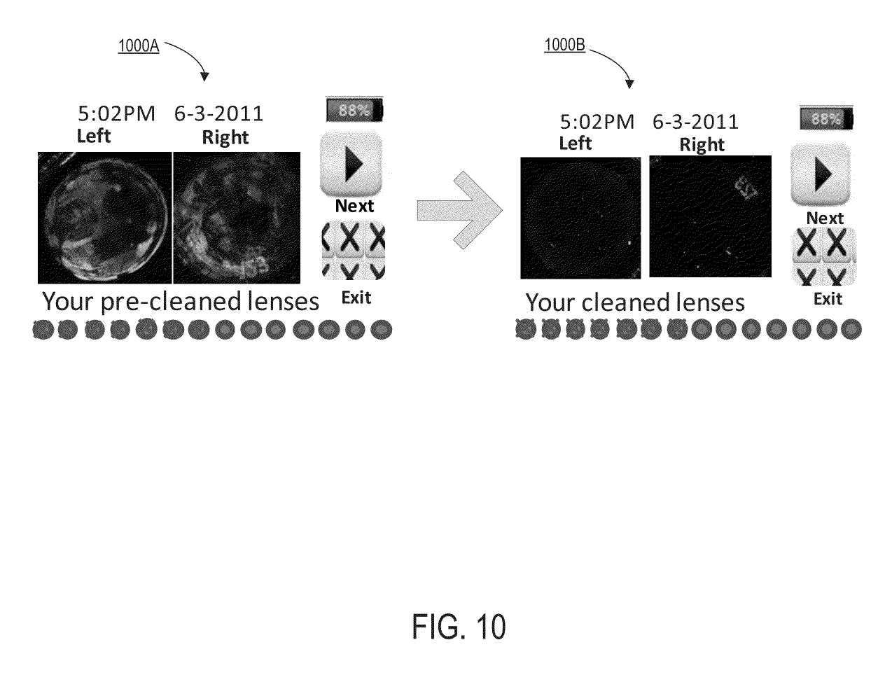

Referring now to FIG. 10, exemplary screen shots of a Ophthalmic Lens Storage Unit device showing a measured pre-cleaned lens and cleaned lens are depicted. At 1000A, the sensors and program may generate an image projected in the touch screen of the Ophthalmic Lens Storage Unit showing a left lens and right lens prior to disinfecting for the user to see. More importantly, the program may take the digital data gathered by the sensors to track the amount of protein buildup or germs present prior to cleaning. This stored digital data may be analyzed to alert the user when there is a change, in protein buildup or specific germs, greater than what would be normal in relation to preprogrammed thresholds. Upon detecting an abnormal amount of protein buildup or germs in the lens, the disinfecting base unit may alert the user, send a picture directly to the eye care practitioner, or cause a specific message to be send to the user through one of the associated network devices discussed in FIG. 8.

Additionally, as depicted the screen shot 1000B the disinfecting base unit can also analyze and generate digital data to determine when the lens is not undergoing the desired cleaning. For example, this may happen when the lens becomes defective, when the lifespan of the lens has ended, or when a lens has expired.

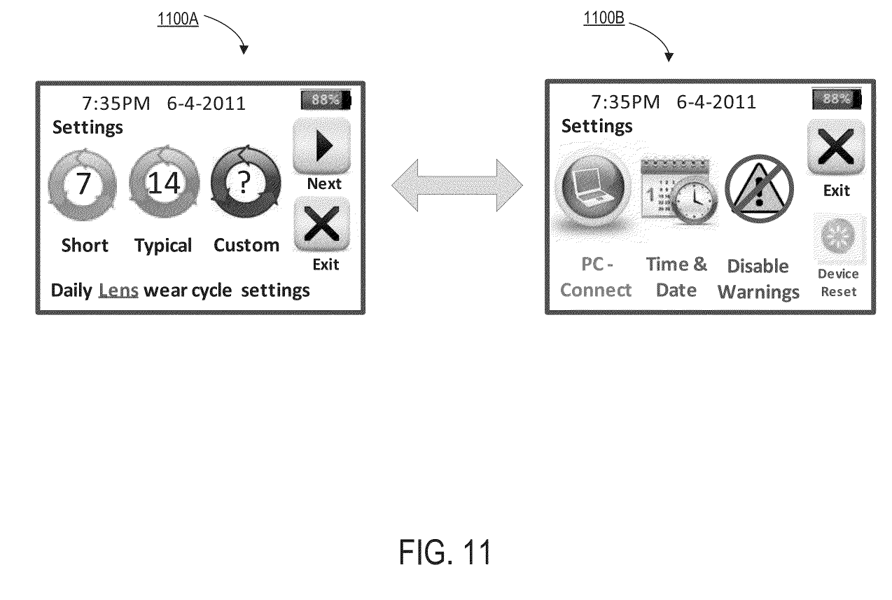

Referring now to FIG. 11 exemplary screen shots of a Ophthalmic Lens Storage Unit depicting additional user settings that can be included in the device are depicted. At 1100A, exemplary settings associated with the lens wear cycle are shown as depicted by the user interphase. These settings can include the user's preferences as to the duration of the cycle. This, as explained in other sections of this disclosure, can cause the program to vary the intensity of the disinfecting radiation, the duration of each dose of radiation and the frequency between said doses of radiation.

At 1100B, user's settings for networks that may be associated with the disinfecting base unit as described in FIG. 8 are included. In addition, time and date user settings which allow the user to change it manually or to link it to a network device, for example, a cell phone, for automatic date/time changes during traveling, or during daytime saving time changes. In addition, weather information and allergy alerts data may be downloaded by the device for message generation and data analysis. Data analysis can include for example, correlating weather and allergy information with abnormal protein or microbial buildup. Warnings may also be controlled to prevent unwanted alerts to the user.

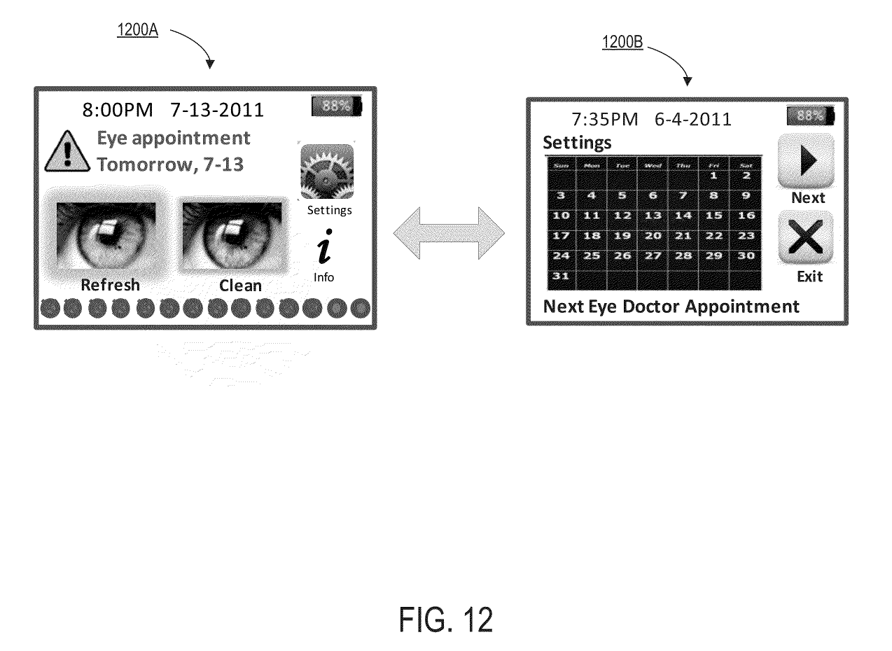

Referring now to FIG. 12, exemplary screen shots of a Ophthalmic Lens Storage Unit associated with Eye Care Practitioner Appointments and Automated Lens Ordering are shown. At 1200A, the screen is showing a reminder to attend a scheduled eye appointment. The disinfecting unit can further cause a network associated device to send additional information about the reminder, for example, directions to the location, cancellation information, etc. After attending the appointment, the user can check off through the interface that he/she has attended the appointment and the disinfecting base unit may record and use this data to track and schedule future appointments. Alternatively, the information can be received from other sources associated with the device. For example, it may be received directly from the eye care practitioner's office, or from a GPS enabled cell phone. Software in the GPS enabled cell phone, for example through the use of Smartphone App can then record and send digital data, to the network or the disinfecting base unit, reflecting that the user was at the location, as scheduled.

Finally, the Ophthalmic Lens Storage Unit may also incorporate changes in the lens prescription, such as for example changes in the prescribed optical power, and include them in the next scheduled automated lens order, or to track that the lenses used from then on are in accordance with the new prescription. Tracking and verification of the lenses may be achieved as a result of, for example, the use the barcode in the lens packaging, or by the data generated from the sensors which may help the programmed software determine the optical power of the particular lens as previously explained.

### Conclusion

The present invention, as described above and as further defined by the claims below, provides methods for collecting, storing and analyzing data for controlling case functions, displaying user messages, and programming an ophthalmic lens disinfecting base unit. Further, communication options allow data to be communicated between the ophthalmic lens storage unit and external devices or entities, through a network, for lens monitoring and automated ordering of lenses.

1. An apparatus for storing one or more ophthalmic Lenses, comprising:
a programmable Ophthalmic Lens Storage Unit for disinfecting one or more ophthalmic lenses, wherein the Ophthalmic Lens Storage Unit includes a processor in logical communication with a digital storage and uses executable software stored in the digital storage and operative with the processor to;
establish a connection between the Ophthalmic Lens Storage Unit and an associated network device, wherein the connection is suitable for communication of digital data and wherein the disinfecting base unit stores one or more ophthalmic lenses;
store a record comprising digital data descriptive of a number of disinfecting cycles the one or more ophthalmic lenses have been subjected to by the disinfecting base unit;
generate an automated order for one or both of: additional ophthalmic lenses and Ophthalmic Lens Storage Unit's components, based upon the stored digital data; and
transmit said automated order via the associated network device.

2. The base unit of claim 1 wherein the order is transmitted based upon receipt of a user's input.

3. The storage unit of claim 1 additionally comprising a sensor for measuring and storing digital data descriptive of a condition of a lens being disinfected.

4. The storage unit of claim 1 additionally comprising a bar code reader capable of downloading and processing corresponding data that identifies a lens product through a network.

5. The storage unit of claim 4 wherein the corresponding data comprises specifications specific to the lens.

6. The storage unit of claim 4 wherein the corresponding data comprises data descriptive of an authenticating feature that can be detected by a sensor in the in the storage unit.

7. The storage unit of claim 6 wherein the storage unit is capable of sending an authentication verification message to the user or a third party upon verification of the authenticating feature in the ophthalmic lens.

8. The base unit of claim 3 wherein the sensor comprises a device to detect one or both of an intensity and a direction of vectors of light to generate data descriptive of optical properties of the ophthalmic lens during disinfection.

9. The base unit of claim 8 wherein the data descriptive of the optical properties of the ophthalmic lens includes optical power and base curve of the lens.

10. The base unit of claim 8 wherein the measured optical properties are compared to the lens specifications to monitor actual performance of the lens.

11. The base unit of claim 8 wherein the actual performance data taken and stored during disinfection for future analysis to detect changes in performance over a set threshold.

12. The base unit of claim 3 wherein the sensor comprises a device to measure a temperature of solution in which the lens is stored.

13. The base unit of claim 1 additionally comprising a user interface capable of displaying message reminders to the user based upon digital data transmitted by the processor.

14. The base unit of claim 1 additionally comprising communication elements capable of sending messages to an associated processing device used for receiving the messages and displaying the messages to one or both the user and a third party.

15. The base unit of claim 1 additionally comprising a means for one or both of the user and an associated 3^{rd} party to input setting preferences for the messages.

16. The base unit of claim 1 additionally comprising a digital storage for storing information related to a disinfecting process.

17. The base unit of claim 1 wherein the processor is capable of using executable software to store and analyze data collected by the sensors, setting preferences and from the executable software analysis.

18. The base unit of claim 13 additionally comprising a universal serial bus connector for providing logical communication between one or both of the processor and the digital storage, and processing device.

19. The base unit of claim 17 wherein the processor is additionally capable of using data collected from additional devices in logical communication with the processor of the base.

20. The base unit of claim 19 wherein the processor is additionally capable of using data collected from additional devices to generate an automated order that can be sent to a 3^{rd} party.

21. The base unit of claim 2 wherein the automated order is send based on the user's predetermined settings.

22. The base unit of claim 2 wherein the automated order is sent upon a confirmation of the user.

23. The base unit of claim 20 wherein the automated order is sent upon a prescription confirmation from an eye care practitioner.

24. The base unit of claim 1 wherein the automated order is for contact lenses.

25. The base unit of claim 1 wherein the automated order is for a disinfecting radiation source.

26. The base unit of claim 1 wherein the automated order is for a disinfecting solution.

27. The base unit of claim 1 additionally comprising an electrical storage for storing power to operate the storage base.

28. The base unit of claim 27 wherein the electrical storage comprises one or more rechargeable batteries.

29. The base unit of claim 27 wherein the electrical storage comprises one or more lithium batteries.
